(19) United States
(12) Patent Application Publication
(10) Pub. No.: US 2015/0359897 A1
(43) Pub. Date: Dec. 17, 2015

(54) METHOD OF TREATING PATIENTS NON-RESPONSIVE TO PALONOSETRON

(71) Applicant: HERON THERAPEUTICS, INC., Redwood City, CA (US)

(72) Inventors: John Barr, Half Moon Bay, CA (US); Erin O'Boyle, Redwood City, CA (US); John B. Whelan, Cupertino, CA (US)

(73) Assignee: HERON THERAPEUTICS, INC., Redwood City, CA (US)

(21) Appl. No.: 14/765,179

(22) PCT Filed: Feb. 4, 2014
(86) PCT No.: PCT/US14/14699

§ 371 Date: Jul. 31, 2015

Related U.S. Application Data
(60) Provisional application No. 61/761,108, filed on Feb. 5, 2013.

(51) Int. Cl. A61K 47/34; A61K 47/10; A61K 9/00; A61K 31/439

(52) U.S. Cl. CPC A61K 47/34; A61K 31/439; A61K 47/10; A61K 9/0024

(57) ABSTRACT

A method for treating chemotherapy-induced nausea and vomiting in individuals undergoing chemotherapy and previously treated with, and whom failed to respond to, a 5-HT3 antagonist other than granisetron is described. Individuals who fail to respond to, for example, palonosetron, as evidenced by an inadequate prevention or attenuation of acute or delayed chemotherapy-induced nausea and vomiting, are treated with a semi-solid drug delivery vehicle that provides a sustained release of granisetron.

### CROSS-REFERENCE TO RELATED APPLICATIONS

This application claims the benefit of priority of U.S. Provisional Patent Application No. 61/761,108, filed Feb. 5, 2013, the content of which is incorporated herein by reference in its entirety.

### TECHNICAL FIELD

The subject matter described herein relates to treatment of chemotherapy-induced nausea and vomiting (CINV) in an individual previously treated with a 5-HT3 receptor modulator, and in particular with the 5-HT3 receptor modulator, palonosetron.

### BACKGROUND

Nausea and vomiting caused by chemotherapy remain among the most distressing side effects for patients undergoing treatment for cancer. Depending upon the chemotherapy agents or regimens given, up to 90% of patients may suffer from some form of chemotherapy-induced nausea and vomiting (CINV) in the absence of antiemetics. Symptoms from CINV are debilitating and can result in some patients refusing further courses of chemotherapy, with obviously unfavorable consequences in regard to progression of the cancer.

CINV is divided into two main categories: acute onset CINV and delayed onset CINV. An additional category, anticipatory CINV, will not be discussed here. Acute CINV typically occurs within the first 24 hours following initial chemotherapeutic treatment; delayed CINV occurs from approximately about 24 hours or more after a course of chemotherapy treatment, often between 24-120 hours following treatment.

Compounds that selectively target 5-hydroxytryptamine 3 (5-HT3) receptors are effective anti-emetics and represent one approach for management of nausea and vomiting in patients undergoing chemotherapy. One 5-HT3 receptor modulator that is administered for preventing or treating both acute and delayed CINV is palonosetron. However, not all patients respond to palonosetron, and there remains a need for treating delayed and acute CINV in these patients, as well as in patients who do not respond to 5-HT3 antagonists other than granisetron.

### BRIEF SUMMARY

The following aspects and embodiments thereof described and illustrated below are meant to be exemplary and illustrative, not limiting in scope.

In one aspect, a method for treating an individual receiving chemotherapy and experiencing or at risk of experiencing chemotherapy-induced nausea and vomiting is provided. More desirably, the method of treatment is directed to an individual receiving chemotherapy and experiencing or at risk of experiencing chemotherapy-induced nausea and vomiting that was not prevented, ameliorated or attenuated by administration of a 5-HT3 receptor antagonist (5-HT3 RA) other than granisetron, such as palonosetron. The method comprises administering, e.g., subcutaneously, a composition comprising a semi-solid delivery vehicle comprised of a bioerodible polymer and granisetron.

In one embodiment, the individual at risk for chemotherapy-induced nausea and vomiting is one who failed to respond to prior treatment with a selective 5-HT3 receptor antagonist other than granisetron, such as, but not limited to, ondansetron, dolasetron, tropisetron, and palonosetron.

In another embodiment of the treatment method, the patient is undergoing treatment for acute chemotherapy-induced nausea and vomiting.

In yet another embodiment, the patient is undergoing treatment for delayed onset chemotherapy-induced nausea and vomiting.

In some cases, the patient is undergoing treatment for both acute and delayed onset chemotherapy-induced nausea and vomiting.

In one embodiment, the patient is undergoing highly emetogenic chemotherapy.

Alternatively, in another embodiment, the patient is undergoing moderately emetogenic chemotherapy.

In an embodiment related to any one or more embodiments as provided herein, the treatment method comprises administering to the patient a single dose of the semi-solid drug delivery vehicle comprising from 1 to 25 mg of granisetron during one cycle of chemotherapy. In a particular embodiment related to the foregoing, the single dose of the semi-solid drug delivery vehicle comprises 5 or 10 mg of granisetron.

In one embodiment related to the foregoing, the single dose is administered prior to commencement of chemotherapy; in an alternative embodiment, the single dose is administered post-chemotherapy.

In yet an additional embodiment, granisetron is the only anti-emetic agent comprised within the semi-solid drug delivery vehicle.

In yet a further embodiment, the treatment method is effective to provide a measurable prevention or reduction of acute or delayed chemotherapy-induced nausea and vomiting when compared to previous treatment with the 5-HT3 antagonist other than granisetron.

In one preferred embodiment, the method is effective to result in a complete absence of an emetic episode in the acute phase.

In yet another preferred embodiment, the method is effective to result in a complete absence of an emetic episode in the delayed phase.

In a further preferred embodiment, the method is effective to result in a complete absence of an emetic episode in both the acute and delayed phase following chemotherapy.

In yet another embodiment, the administering is continued over one or more additional cycles of chemotherapy.

In one or more further embodiments related to the bioerodible polymer comprised within the semi-solid delivery vehicle, the bioerodible polymer is a polyorthoester.

In a particular embodiment of the method, the polyorthoester comprises subunits selected from

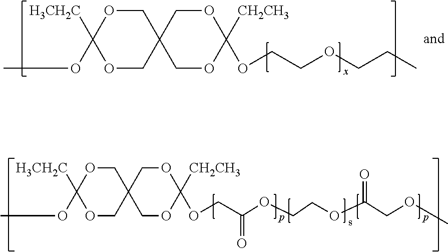

where

x is an integer selected from 1, 2, 3, and 4,

the total amount of p is an integer selected from 1, 2, 3, 4, 5, 6, 7, 8, 9, 10, 11, 12, 13, 14, 15, 16, 17, 18, 19 and 20,

s is an integer selected from 1, 2, 3, and 4,

the mole percentage of α-hydroxyacid containing subunits in the polyorthoester is from about 0.1 to about 25 mole percent,

and the polyorthoester has a molecular weight in a range of about 1000 to 10,000.

In yet another embodiment, the polyorthoester comprised in the semi-solid drug delivery vehicle is a reaction product of 3,9-di(ethylidene)-2,4,8,10-tetraoxaspiro[5.5]undecane (DETOSU), triethylene glycol and triethylene glycol diglycolide.

In one or more additional embodiments, the mole percentage of glycolide-containing subunits in the polyorthoester is from about 0.1 to about 25 mole percent.

In a further embodiment of the method, the semi-solid drug delivery vehicle comprises an excipient, selected from, for example, a polyethylene glycol ether derivative with a molecular weight of between about 200-4,000. One example is a polyethylene glycol monomethyl ether, such as polyethylene glycol monomethyl ether (mPEG) 550. Other polyethylene glycol mono- and di-alkyl ethers are contemplated.

In yet a more specific embodiment, the semi-solid drug delivery vehicle comprises a polyorthoester, about 10-50 weight percent polyethylene glycol monomethyl ether having a molecular weight in a range of about 200 to 4,000, and about 1-5 weight percent granisetron. In yet another embodiment, the semi-solid drug delivery vehicle comprises from about 70-80 weight percent polyorthoester, about 15-25 weight percent polyethylene glycol monomethyl ether and from 1 to 5 weight percent granisetron.

Also provided herein is a semi-solid drug delivery vehicle comprising a bioerodible polymer and granisetron, for use in treatment of acute or delayed chemotherapy-induced nausea and vomiting in a patient undergoing chemotherapy, wherein the patient was previously treated with a 5-HT3 antagonist other than granisetron and failed to achieve a satisfactory prevention or reduction of acute or delayed chemotherapy-induced nausea and vomiting.

In addition to the exemplary aspects and embodiments described above, further aspects and embodiments will become apparent by reference to the data presented herein and by study of the following descriptions, examples, and claims.

As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment of the present invention. Additional aspects and advantages of the present invention are set forth in the following description and claims, particularly when considered in conjunction with the accompanying examples.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### DETAILED DESCRIPTION

### I. Definitions

Unless defined otherwise in this specification, all technical and scientific terms are used herein according to their conventional definitions as they are commonly used and understood by those of ordinary skill in the art of synthetic chemistry, pharmacology and medicine.

Where a range of values is provided, it is intended that each intervening value between the upper and lower limit of that range and any other stated or intervening value in that stated range is encompassed within the disclosure. For example, if a range of 1 to 8 is stated, it is intended that 2, 3, 4, 5, 6, and 7 are also explicitly disclosed, as well as the range of values greater than or equal to 1 and the range of values less than or equal to 8.

As used in this specification, the singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

A “polymer susceptible to hydrolysis” refers to a polymer that is capable of degradation, disassembly or digestion through reaction with water molecules. Such a polymer contains hydrolyzable groups, such as an ester group, in the polymer. Examples of polymers susceptible to hydrolysis may include, but is not limited to, polyorthoester, such as those described herein, and those described in U.S. Pat. Nos. 4,079,038, 4,093,709, 4,131,648, 4,138,344, 4,180,646, 4,304,767, 4,957,998, 4,946,931 and 5,968,543, and U.S. Patent Publication No. 2007/0265329, which are incorporated by reference in their entirety.

“Bioerodible” and “bioerodibility” refer to the degradation, disassembly or digestion of a polymer by action of a biological environment, including the action of living organisms and most notably at physiological pH and temperature. As an example, a principal mechanism for bioerosion of a polyorthoester is hydrolysis of linkages between and within the units of the polyorthoester.

“Semi-solid” denotes the mechano-physical state of a material that is flowable under moderate stress. More specifically, the semi-solid material should have a viscosity between about 10,000 and 3,000,000 cps, especially between about 30,000 and 500,000 cps. Preferably the composition or formulation is easily syringable or injectable, meaning that it can readily be dispensed from a conventional tube of the kind well known for topical or ophthalmic formulations, from a needleless syringe, or from a syringe with a 16 gauge or smaller needle, such as 16-25 gauge.

The term, “delivery vehicle”, denotes a composition which has functions including transporting an active agent to a site of interest, controlling the rate of access to, or release of, the active agent by sequestration or other means, and facilitating the application of the agent to the region where its activity is needed.

“Molecular mass” in the context of a polymer such as a polyorthoester, refers to the nominal average molecular mass of a polymer, typically determined by size exclusion chromatography, light scattering techniques, or velocity. Molecular weight can be expressed as either a number-average molecular weight or a weight-average molecular weight. Unless otherwise indicated, all references to molecular weight herein refer to the weight-average molecular weight. Both molecular weight determinations, number-average and weight-average, can be measured using gel permeation chromatographic or other liquid chromatographic techniques. Other methods for measuring molecular weight values can also be used, such as the measurement of colligative properties (e.g., freezing-point depression, boiling-point elevation, or osmotic pressure) to determine number-average molecular weight or the use of light scattering techniques, ultracentrifugation or viscometry to determine weight-average molecular weight. The polymers of the invention are typically polydisperse (i.e., number-average molecular weight and weight-average molecular weight of the polymers are not equal), possessing low polydispersity values such as less than about 1.2, less than about 1.15, less than about 1.10, less than about 1.05, and less than about 1.03.

“Pharmaceutically acceptable salt” denotes a salt form of a drug having at least one group suitable for salt formation that causes no significant adverse toxicological effects to the patient. Pharmaceutically acceptable salts include salts prepared by reaction with an inorganic acid, an organic add, a basic amino add, or an acidic amino acid, depending upon the nature of the functional group(s) in the drug. Suitable pharmaceutically acceptable salts include acid addition salts which may, for example, be formed by mixing a solution of a basic drug with a solution of an acid capable of forming a pharmaceutically acceptable salt form of the basic drug, such as hydrochloric acid, iodic acid, fumaric acid, maleic acid, succinic acid, acetic acid, citric acid, tartaric acid, carbonic acid, phosphoric acid, sulfuric acid and the like. Typical anions for basic drugs, when in protonated form, include chloride, sulfate, bromide, mesylate, maleate, citrate and phosphate. Suitably pharmaceutically acceptable salt forms are found in, e.g., Handbook of Pharmaceutical Salts: Properties, Selection and Use, Weinheim/Zürich:Wiley-VCH/VHCA, 2002; P. H. Stahl and C. G. Wermuth, Eds.

“Treating” or “treatment” of a disease or condition includes preventing the disease or condition from occurring in an animal that may be predisposed to the disease or condition but does not yet experience or exhibit symptoms of the disease (prophylactic treatment), inhibiting the disease (slowing or arresting its development), providing relief from the symptoms or side-effects of the disease or condition (including palliative treatment), and relieving the disease (causing regression of the disease). For the purposes of the embodiments described herein, one condition that may be treated is chemotherapy induced nausea and vomiting or CINV.

A patient that has been previously treated with a 5-HT3 receptor antagonist and “failed to achieve a satisfactory prevention or reduction of acute or delayed CINV”, also referred to herein as being “unresponsive to treatment”, is one that has been administered a 5-HT3 receptor antagonist at a recommended dosage amount, route of administration and dosing regimen, as provided in the corresponding 5-HT3 receptor antagonist drug label, where the previous treatment failed to provide an absence of emetic episodes over a specified time period, e.g., 0-24 hours after chemotherapy (acute phase) and/or 24 to 120 hours after chemotherapy (delayed phase), in either a first round, or subsequent rounds of chemotherapy.

A semi-solid composition that is “effective to provide a measurable prevention or reduction of acute or delayed chemotherapy-induced nausea or vomiting when compared to previous treatment with a 5-HT3 receptor antagonist” [to which the patient was unresponsive] is one that, when administered at a therapeutically effective dose, provides a prevention or reduction of CINV that is improved over that experienced by the patient when treated as recommended with the prior 5-HT3 receptor antagonist (to which the patient was unresponsive). Thus, if recommended treatment with the prior 5-HT3 receptor antagonist results in a certain number of emetic episodes over the specified time period, a semi-solid composition that is improved is one that produces a fewer number of emetic episodes over the same specified time period. In a most preferred situation, administration of the semi-solid composition is effective to provide an absence of emetic episodes over the specified time period, in either a first cycle, or subsequent cycles of chemotherapy.

Optional” or “optionally” means that the subsequently described circumstance may or may not occur, so that the description includes instances where the circumstance occurs and instances where it does not.

The term “substantially” in reference to a certain feature or entity means to a significant degree or nearly completely (i.e. to a degree of 85% or greater) in reference to the feature or entity.

The term “about”, particularly in reference to a given quantity, is meant to encompass deviations of plus or minus five percent.

Additional definitions may also be found in the sections which follow.

### II. Method of Treatment

The idealized goal of antiemetic therapy is the complete prevention of CINV, i.e., nausea and/or vomiting that is associated with chemotherapeutic treatment of a patient diagnosed with cancer. While it is desirable to provide prophylactic treatment to prevent any onset of nausea and/or vomiting, the present disclosure is understood to also include treatment after onset of symptoms such as nausea and/or vomiting.

The present method is based, at least in part, upon the discovery that certain subjects undergoing treatment for CINV by administration of the 5-HT3 receptor antagonist, palonosetron, were found to be unresponsive. These subjects were then treated with another 5-HT3 receptor antagonist, granisetron, comprised within a semi-solid polyorthoester-based drug delivery vehicle, and surprisingly, responded favorably to such anti-emetic treatment. Interestingly, palonosetron is a second generation 5-HT3 receptor antagonist and is generally considered to be more effective in treating CINV when compared to the first generation 5-HT3 receptor antagonists, including granisetron (to be described in greater detail below). Thus, in general, the treatment method provided herein comprises administering to a patient undergoing either high or moderate emetic risk chemotherapy, a semi-solid drug delivery vehicle comprising a bioerodible polymer and granisetron, where the patient was previously treated with a 5-HT3 antagonist other than granisetron and failed to achieve a satisfactory prevention or reduction of acute or delayed chemotherapy-induced nausea and vomiting.

### A. General Emetic Classifications

The incidence and severity of emesis in patients receiving chemotherapy varies according to many factors. These factors include the particular chemotherapeutic agent(s) administered, dose, schedule of administration, route, and individual patient variables. As previously described, emesis is generally classified as acute, occurring 0 (i.e., immediately) to 24 hours post-chemotherapy, or delayed, occurring 24 to 120 hours following chemotherapy. A subject undergoing chemotherapy may experience both acute and delayed emesis.

Chemotherapy is generally stratified/classified according to the degree of emesis that is typically associated with the chemotherapy. Classifications include highly emetogenic chemotherapy, moderately emetogenic chemotherapy, and low potential or minimal risk regimens. This stratification was developed by the American Society of Clinical Oncology for chemotherapeutic agents and their respective risk of acute and delayed emesis (Kris, M. G., Hesketh, P. J., Somerfield, M. R., et al.: American Society of Clinical Oncology Guideline for Antiemetics in Oncology: Update 2006. J Clin Oncol 24 (18): 2932-47, 2006). The examples of chemotherapeutic agents currently falling within various emetic classifications as provided below is meant to be exemplary, since drugs falling within a given classification, can, in certain instances, change.

Typically, high emetogenic chemotherapy regimens cause CINV more than 90% of the time. That is to say, for highly emetogenic agents, nearly every patient administered the agent would vomit if an antiemetic agent was not administered. Examples of chemotherapeutic agents that are currently classified as highly emetogenic include, among others, cisplatin, and cyclophosphamide at doses >1,500 mg/m². Other examples of highly emetogenic agents include mechlorethamine, streptozotocin, carmustine, dacarbazine, dactinomycin, lomustine and pentostatin. Thus, the emetogenic potential for patients treated with an agent falling within this class, in the absence of anti-emetic therapy, is the highest.

Moderately emetogenic chemotherapeutic agents are those which cause CINV 30%-90% of the time in the absence of an anti-emetic. Moderately emetogenic agents include, for example, oxaliplatin, carboplatin, the anthracyclines, such as daunorubicin, doxorubicin, epirubicin, and idarubicin; cytarabine at doses greater than 1 g/m², ifosfamide, cyclophosphamide at doses <1,500 mg/m², irinotecan, alretamine, melphalan, mitoxantrone, temozolamide, trabectedin, and treosulfan.

Further classifications include low and minimal emetic risk chemotherapeutic agents. Low-risk agents are those which cause CINV 10%-30% of the time, while minimal risk anti-cancer agents cause CINV less than 10% of the time. Examples of low risk chemotherapeutic agents include taxanes such as docetaxel and paclitaxel; mitoxantrone, gemcitabine, 5-fluorouracil, mitomycin, topotecan, etoposide, pemetrexed, methotrexate, cytarabine, bortezomib, cetuximab, and trastuzumab. Examples of currently classified minimal risk chemotherapeutic agents include bleomycin, busulfan, 2-chlorodeoxyadenosine, fludarabine, the vinca alkaloids (vinorelbine, vinblastine, and vincristine), and bevacizumab (Hawkins, R. et al., Clinical J. Oncology Nursing, 13(1): 54-64 (2009)). Additional chemotherapeutics associated with a minimal emetogenic risk include, for example, rituximab, bleomycin, busulphan, fludarabine, and 2-chlorodeoxyadenosine.

One can therefore assess whether a chemotherapeutic agent is considered to be highly or moderately emetogenic based upon its associated degree of emesis as described above, and in, e.g., Jordan, K., et al., The Oncologist, 2007; 12:1143-1150, along with references cited therein.

### B. 5-HT₃ Receptor Antagonists

In the current method, the patient is one who is undergoing chemotherapy and was unresponsive to prior anti-emetic therapy when treated with a therapeutically effective amount of a 5-HT3 receptor antagonist other than granisetron such as palonosetron. For instance, the patient may have been previously treated with, e.g., ondansetron, dolasetron, palonosetron, or tropisetron, by one of the recommended dosing regimens described below, or as described in the label instructions of the dosage form of the drug employed. Drugs falling within this category possess a high therapeutic index for the management of CINV. The 5-HT3 receptor antagonists prevent nausea and vomiting by preventing serotonin, which is released from enterochromaffin cells in the gastrointestinal mucosa, from initiating afferent transmissions to the central nervous system via vagal and spinal sympathetic nerves (Tyers, M. B., Semin Oncol 19 (4 Suppl 10): 1-8, 1992). Serotonin receptor antagonists used to treat CINV include ondansetron, granisetron, dolasetron, and palonosetron. Another serotonin receptor antagonist, tropisetron, while not currently approved by the FDA, is available internationally. Of the foregoing, first generation 5-HT3 receptor antagonists include ondansetron, granisetron, dolasetron, and tropisetron. Palonosetron is a second generation 5-HT3 receptor antagonist. Palonosetron is highly selective, and has a longer half-life and greater receptor binding affinity versus the first generation 5-HT3RAs (Smith, H. S., et al., Ann Paffiat Med, 2012; 1 (2): 115-120). Exemplary 5-HT3 receptor antagonists with which a patient may have been previously treated and illustrative dosing regimens are described below.

A patient as described herein may have been previously treated with and unresponsive to anti-emetic treatment with the 5-HT3RA, ondansetron, e.g., when administered either orally or via injection. For example, the patient may have been administered ondansetron at a dosage of 0.15 mg/kg intravenously 15 to 30 minutes prior the chemotherapy. When following the recommended therapy, the dose is repeated every 4 hours for two additional doses for a total of three doses. Alternatively, oral ondansetron may have been administered 3 times daily starting 30 minutes before chemotherapy and continuing for up to 2 days post-chemotherapy, at a dosage amount of 4 mg per dose. When administered for prevention of emesis associated with highly emetogenic chemotherapy, a recommended adult oral dosage of ondansetron is 24 mg administered as three 8-mg tablets approximately 30 minutes before the start of chemotherapy. When used for the prevention of nausea and vomiting associated with moderately emetogenic cancer chemotherapy, the recommended adult oral dosage of ondansetron is one 8-mg tablet or one 8-mg oral-disintegrating tablet, or 10 mL of oral solution given twice a day. Recommended dosage forms and dosage regimens for an anti-emetic 5-HT₃ receptor antagonist such as ondansetron, or any of the 5-HT3 receptor antagonists described, are provided in the label instructions for the corresponding branded form of the drug. The dosing regimens described herein are meant to be exemplary, and indicate that numerous variations exist in dosage forms, formulations, and dosing regimens, depending upon the cancer being treated, the associated emetogenic risk, the chemotherapeutic agent, patient considerations, etc.

Another 5-HT3 receptor antagonist that may have been previously administered to a patient for the treatment of CINV is dolasetron. The dolasetron may have been administered, for example, either orally or as an injectable dosage. For example, the patient may have been dosed orally with 100 mg of dolasetron within one hour prior to commencement of chemotherapy. Alternatively, for example, the patient may have been administered dolasetron intravenously or orally at 1.8 milligrams/kilogram as a single dose approximately 30 minutes prior to chemotherapy.

Similarly, the patient may have been administered tropisetron according to recommended dosing procedures.

In a preferred embodiment, the 5-HT₃ receptor antagonist with which a patient has been previously treated for CINV is palonosetron. As discussed above, palonosetron is a second generation 5-HT₃ receptor antagonist, and has been reported to possess, when compared to first generation 5-HT3 receptor antagonists such as dolasetron, granisetron and ondansetron, a higher binding affinity to the 5-HT₃ receptors, a higher potency, a significantly longer half-life, and an excellent safety profile (Eisenberg, P.; MacKintosh, F. R.; Ritch, P., et al., Ann Oncol 15 (2): 330-7, 2004). For example, the patient may have been administered a single 0.25 mg intravenous dose of palonosetron approximately 30 minutes prior to the start of chemotherapy. Palonosetron is typically administered for the prevention of acute CINV associated with initial and repeat courses of both moderately and highly emetogenic cancer chemotherapy, and for the prevention of delayed CINV associated with initial and repeat courses of moderately emetogenic cancer chemotherapy.

In accordance with the instant disclosure, a patient that has been previously treated with a 5-HT3 receptor antagonist such as palonosetron that is unresponsive to treatment is one that has been administered the 5-HT3 receptor antagonist at a recommended dosage amount, route of administration and dosing regimen, for example, as provided in the 5-HT3 receptor antagonist drug label, where the previous treatment failed to provide an absence of emetic episodes over a specified time period, e.g., 0-24 hours after chemotherapy (acute) and/or 24 to 120 hours after chemotherapy (delayed), in either a first round, or subsequent rounds of chemotherapy.

Prior anti-emetic treatment of the patient with any of the above 5-HT3 receptor antagonists may have been for a patient undergoing highly emetogenic chemotherapy or moderately emetogenic chemotherapy.

### C. Patient Population

The instant method is directed to a method for treating a patient for chemotherapy-induced nausea and vomiting (CINV). The patient undergoing treatment is one who was previously treated with a 5-HT3 antagonist other than granisetron and failed to achieve a satisfactory prevention or reduction of acute or delayed chemotherapy-induced nausea and vomiting. The 5-HT3 receptor antagonists other than granisetron with which the patient may have been previously treated, and to which treatment the patient was unresponsive, include ondansetron, dolasetron, palonosetron, and tropisetron. In a preferred and illustrative embodiment of the method, the 5-HT3 receptor antagonist is palonosetron, e.g., administered intravenously. As discussed previously, a patient that was determined to be unresponsive to treatment is one in which the recommended anti-emetic treatment failed to provide an absence of emetic episodes over a specified time period.

The present method includes the step of administering to a patient undergoing chemotherapy, a semi-solid drug delivery vehicle comprising a bioerodible polymer and granisetron. Patients include males, females, adults, pediatric patients, and elderly patients. In one embodiment, the patient is one at greater risk for experiencing emesis. Such patients include females, females who have experienced emesis during pregnancy, and those with a history of low alcohol intake.

### D. Drug-Delivery Vehicle

The drug-delivery vehicle contemplated for the treatment method described herein is, in one embodiment, comprised of a bioerodible polymer and a 5-HT3 antagonist such as granisetron. Exemplary vehicles are described in U.S. Pat. No. 8,252,304 and U.S. Patent Application Publication No. 2007/0264338, which are incorporated by reference herein. In one embodiment, the vehicle is comprised of a polyorthoester, and an exemplary vehicle is set forth in Example 1. The semi-solid delivery vehicle provides for controlled release of the granisetron contained therein.

Semi-solid polyorthoester polymers are generally prepared by condensation reactions between diketene acetals and polyols, preferably diols, to provide polymers having differences in their mechanophysical state and bioerodibility, based upon the selection of the diol component(s), to be explained in greater detail below.

Exemplary polyorthoesters for use in the compositions provided herein possess a molecular weight of about 1,000 Da to 20,000 Da, for example from 1,000 Da to 10,000 Da or from 1,000 Da to 8,000 Da, or from about 1,500 Da to about 7,000 Da.

Polyorthoesters that can be utilized in the presently disclosed semi-solid compositions are selected from formulas I and II below:

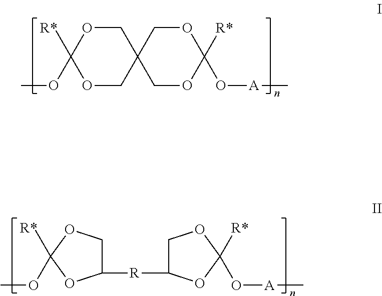

where:
R is a bond, —(CH₂)ₐ—, or —(CH₂)_{b}—O—(CH₂)_{c}—; where a is an integer from 1 to 10, and b and c are independently integers from 1-5; R* is a C₁₋₄ alkyl;
R⁰, R^{II} and R^{III} are each independently H or C₁₋₄ alkyl;
n is an integer of at least 5, for example, from 5 to 1000; and
A is R¹, R², R³, or R⁴, where
R¹ is:

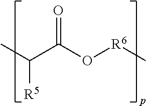

where:
p is an integer of 1 to 20;
R⁵ is hydrogen or C₁₋₄ alkyl; and
R⁶ is:

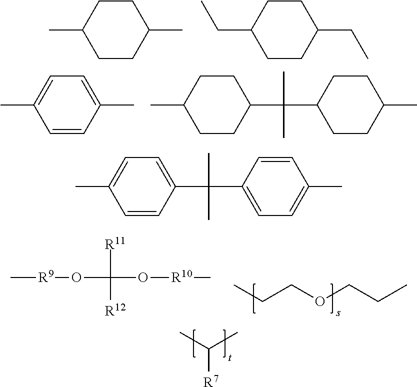

where:
s is an integer of 0 to 30;
t is an integer of 2 to 200; and
R⁷ is hydrogen or C₁₋₄ alkyl;
R² is:

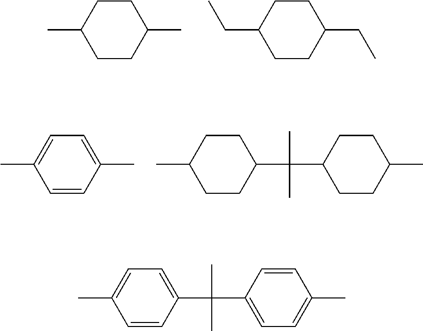

R³ is:

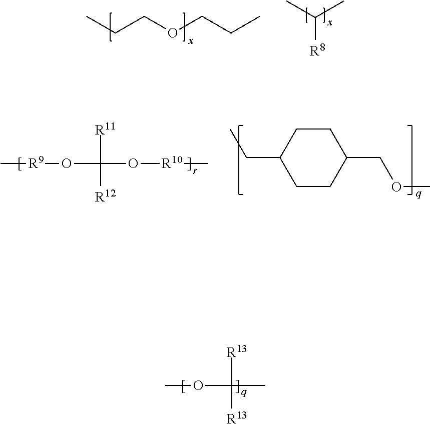

where:

x is an integer of 0 to 100;

y is an integer of 2 to 200;

q is an integer of 2 to 20;

r is an integer of 1 to 20;

R⁹ is hydrogen or C₁₋₄ alkyl;

R⁹ and R¹⁰ are independently C₁₋₁₂ alkylene;

R¹¹ is hydrogen or C₁₋₆ alkyl and R¹² is C₁₋₆ alkyl; or R₁₁ and R₁₂ together are C₃₋₁₀ alkylene; and

R⁴ is the residue of a diol containing at least one functional group independently selected form amide, imide, urea, and urethane groups;

in which at least 0.01 mol percent of the A units are of the formula R¹.

In one preferred embodiment, the polyorthoester is described by formula I.

The polyorthoester polymers are prepared, for example, by reaction of a diketene acetal according to one of the following formulas:

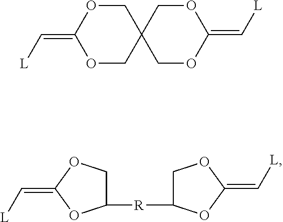

where L is hydrogen or a C₁₋₃ alkyl, and R is as defined above, with a diol according to formula HO—R¹—OH and at least one diol according to the formulae, HO—R²—OH, HO—R³—OH, or HO—R⁴—OH (where (where R¹, R², R³ and R⁴ are as described above). In the presence of water, the α-hydroxy acid containing subunits are readily hydrolyzed at body temperature and at physiological pH to produce the corresponding hydroxyacids, which can then act as catalysts to control the hydrolysis rate of the polyorthoester without the addition of exogenous acid. Thus, polyorthoesters having a higher mole percentage of α-hydroxy acid containing subunits possess a higher degree of bioerodibility.

Preferred polyorthoesters are those in which the mole percentage of α-hydroxy acid containing subunits is at least about 0.01 mole percent. Exemplary percentages of α-hydroxy acid containing subunits in the polymer (e.g., glycolide-derived subunits) are from about 0.01 to about 50 mole percent, preferably from about 0.05 to about 30 mole percent, from about 0.1 to about 25 mole percent. As an illustration, the percentage of α-hydroxy acid containing subunits may be 1, 2, 3, 4, 5, 6, 7, 8, 9, 10, 11, 12, 13, 14, 15, 16, 17, 18, 19, 20, 21, 22, 23, 24, 24, 26, 27, 28, 29 or 30 mol percent, including any and all ranges lying therein, formed by combination of any one lower mole percentage number with any higher mole percentage number.

Particularly preferred polyorthoesters are those in which R⁵ is hydrogen or methyl; R⁶ is

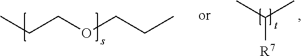

where s is an integer from 0 to 10, e.g., preferably selected from 1, 2, 3, or 4; t is an integer from 2 to 30, particularly selected from 2, 3, 4, 5, 6, 7, 8, 9 and 10; R⁷ is hydrogen or methyl; and R³ is

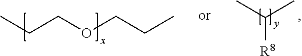

where x is an integer from 0 to 10, e.g., preferably selected from 1, 2, 3, or 4; y is an integer from 2 to 30, particularly selected from 2, 3, 4, 5, 6, 7, 8, 9 and 10; R⁸ is hydrogen or methyl; R⁴ is selected from a residue of an aliphatic diol having from 2-20 carbon atoms (e.g., selected from 2, 3, 4, 5, 6, 7, 8, 9, 10, 11, 12, 13, 14, 15, 16, 17, 18, 19, and 20 carbon atoms), preferably having from 2 to 10 carbon atoms, interrupted by by one or two amide, imide, urea, or urethane groups. Preferably, the proportion of subunits in the polyorthoester in which A is R¹ is from about 0.01-50 mole percent, more preferably from about 0.05 to about 30 mole percent, and even more preferably from about 0.1 to 25 mole percent. Illustrative and preferred mole percentages include 10, 15, 25 and 25 mole percent of percentage of subunits in the polyorthoester in which A is R¹. In one preferred embodiment, the mole percent is 20. Additionally, typically, the proportion of subunits in which A is R² is less than 20 percent, preferably less than about 10 percent, and more preferably less than about 5 percent, and the proportion of subunits in which A is R⁴ is less than 20 percent, preferably less than about 10 percent and more preferably less than 5 percent.

An exemplary and preferred polyorthoester comprises subunits selected from

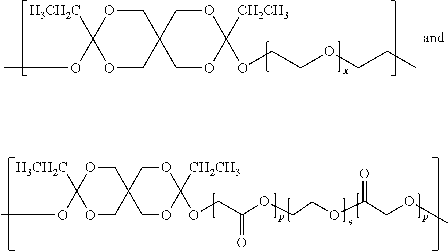

where
x is an integer from 1-4 (e.g., can be selected from 1, 2, 3, and 4) the total amount of p is an integer from 1-20 (e.g., can be selected from 1, 2, 3, 4, 5, 6, 7, 8, 9, 10, 11, 12, 13, 14, 15, 16, 17, 18, 19, and 20),
s is an integer from 1-4 (e.g., can be selected from 1, 2, 3, and 4),
the mole percentage of α-hydroxyacid containing subunits in the polyorthoester is from about 0.1 to about 25 mole percent, and the polyorthoester has a molecular weight in a range of about 1,000 Da to 10,000 Da.

For example, in one embodiment, the polyorthoester comprises alternating residues of 3,9-diethyl-3,9-2,4,8,10-tetraoxaspiro[5.5]undecane-3,9-diyl:

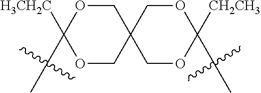

and a diol-ate residue of triethylene glycol or of triethylene glycol diglycolide prepared by reacting triethylene glycol with from 0.5 to 10 molar equivalents of glycolide at about 100-200° C. for about 12 hours to 48 hours. Typically, the mole percentage of glycolide-containing subunits in the polyorthoester is from about 0.1 to about 25 mole percent, and the polyorthoester has a molecular weight of about 1,000 Da to 10,000 Da.

Polyorthoesters such as those described above are prepared by reacting an illustrative diketene acetal, 3,9-di(ethylidene)-2,4,8,10-tetraoxaspiro[5.5]undecane (DETOSU),

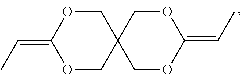

with one or more diols as described above, e.g., triethylene glycol (TEG) and triethylene glycol diglycolide (TEGdiGL). Diols such as triethylene diglycolide or triethylene monoglycolide, or the like, are prepared as described in U.S. Pat. No. 5,968,543, e.g., by reacting triethylene glcol and glycolide under anhydrous conditions to form the desired product. For example, a diol of the formula HO—R¹—OH comprising a polyester moiety may be prepared by reacting a diol of the formula HO—R⁶—OH with between 0.5 and 10 molar equivalents of a cyclic diester of an α-hydroxy acid such as lactide or glycolide, and allowing the reaction to proceed at 100-200° C. for about 12 hours to about 48 hours. Suitable solvents for the reaction include organic solvents such as dimethylacetamide, dimethyl sulfoxide, dimethylformamide, acetonitrile, pyrrolidone, tetrahydrofuran, and methylbutyl ether. Although the diol product is generally referred to herein as a discrete and simplified entity, e.g., TEG diglycolide (and products such as TEG diglycolide), it will be understood by those of skill in the art that due to the reactive nature of the reactants, e.g., ring opening of the glycolide, the diol is actually a complex mixture resulting from the reaction, such that the term, TEG diglycolide, generally refers to the average or overall nature of the product. In a preferred embodiment, the polyorthoester is prepared by reacting DETOSU, triethylene glycol, and triethylene glycol diglycolide in the following molar ratios: 90:80:20. Thus, in a particularly preferred embodiment, the polyorthoester comprises about 20 mole percent R¹, where R¹ is triethylene glycol diglycolide, and 80 mole percent R³, where R³ is triethylene glycol.

The semi-solid compositions provided herein typically contain one or more excipients. Preferably, the excipient is a pharmaceutically-acceptable polyorthoester compatible liquid excipient. Such excipients are liquid at room temperature and are readily miscible with polyorthoesters. Exemplary polyorthoester compatible liquid excipients include polyethylene glycol having a molecular weight between about 200 Da and 4,000 Da, or a polyethylene glycol derivative or co-polymer having a molecular weight between about 200 Da and 4,000 Da, e.g., an end-capped PEG such as monomethoxypolyethylene glycol, or a mono-, di- or triglyceride of a C2-19 aliphatic carboxylic acid or a mixture of such acids, alkoxylated tetrahydrofurfuryl alcohols and their C1-C4 alkyl ethers, dimethyl sulfoxide (DMSO), and C2-19 aliphatic carboxylic acid esters, or the like. A preferred excipient is monomethoxy-PEG, having a molecular weight selected from 400, 450, 500, 550, 600 and 650.

In another embodiment, the pharmaceutically-acceptable polyorthoester compatible liquid is an aprotic solvent. Compositions/drug delivery vehicles suitable for use in the instant method may comprise a polyorthoester, an aprotic solvent, and granisetron, as described in U.S. Provisional Patent Application No. 61/789,469, filed Mar. 15, 2013, the content of which is incorporated herein by reference. The solvent can be either water miscible, partially water miscible, or poorly water miscible, depending on the desired release profile for a given active agent and the solubility of the active agent in the polyorthoester polymer and polymer/solvent combination. Suitable hydrophilic (water miscible) biocompatible organic solvents that may be used have, in one embodiment, water solubility greater than 10% by weight of the solvent in water. Examples of such hydrophilic biocompatible organic solvents include amides such as N-methyl-2-pyrrolidone (NMP), 2-pyrrolidone, N-ethyl-2-pyrrolidone, N-cycylohexyl-2-pyrrolidone, dimethyl acetamide, and dimethyl formamide; esters of monobasic acids such as methyl lactate, ethyl lactate, and methyl acetate; sulfoxides such as dimethyl sulfoxide and decylmethylsulfoxide; lactones such as e-caprolactone and butyrolactone; ketones such as acetone and methyl ethyl ketone; and ethers such as dimethyl isosorbide and tetrahydrofuran. Suitable lipophilic biocompatible organic solvents that may be used in the compositions and delivery systems described herein have, in one embodiment, a water solubility less than 10% by weight of the solvent in water. Examples of such lipophilic biocompatible organic solvents include esters of mono-, di-, and tricarboxylic acids such as ethyl acetate, ethyl oleate and isopropyl myristate; and esters of aromatic acids such as benzyl benzoate. Combinations of different hydrophilic solvents can be used to obtain higher or lower levels of solubility of the liquid polymer and bioactive agent in the resultant solution. A combination of organic solvents can also be used to control the rate of release of an active agent such as granisetron by controlling the rate at which the solvent dissolves or dissipates when the liquid polymer/solvent/active agent composition is placed in the body.

The semi-solid composition, sometimes referred to as a delivery vehicle, is typically prepared by mixing or blending the polyorthoester and the polyorthoester-compatible liquid. The mixing or blending can be performed by any suitable method, generally at a temperature less than about 50° C., e.g., at room temperature, although in certain instances, depending upon the nature of the materials, mixing or blending may be carried out at higher temperatures, e.g., from about 25 to 100° C. The mixing or blending is generally carried out in the absence of solvents, to obtain a homogeneous, flowable and non-tacky semi-solid formulation at room temperature.

Granisetron is generally mixed with the semi-solid composition in the same manner as which it was formed, i.e., by conventional blending. The blending is generally carried out in a fashion suitable to obtain a homogeneous distribution of the components in the formulation, i.e., by mixing the components in any order necessary to achieve homogeneity. It is preferred that the particle size of the granisetron is sufficiently small (e.g., 1-100 microns, or preferably, from 5-50 microns), to provide a resulting composition that is smooth; typically the granisetron is milled into fine particles preferably less than 100 microns in size and sieved before mixing with the other semi-solid components. The granisetron may be mixed with the semi-solid composition that has already been formed or can be mixed together with the polyorthoester and polyorthoester-compatible liquid to form the final semi-solid composition. The components, including the granisetron, are mixed in any order to achieve a homogeneous composition.

A preferred semi-solid composition contains a polyorthoester, polyethylene glycol monomethylether 550 (also referred to as mPEG or monomethoxy PEG), and granisetron, where the polyorthoester is prepared by reaction of DETOSU:TEG:TEG-diGL, at relative molar ratios of 90:80:20. The relative concentrations of the components of the semi-solid composition will vary depending upon the amount of active agent(s), polyorthoester, and polyorthoester-compatible liquid. The weight percent of the polyorthoester compatible liquid can range from about 10-50 weight percent, or from about 10-40 weight percent, or from 10-30 weight percent, or from 10-25 weight percent. Exemplary amounts are about 10, 12, 15, 20, 25, 30, 35, 40, 45 or 50 weight percent of the polyorthoester-compatible liquid such as mPEG 550 or any other suitable polyorthoester-compatible liquid as described previously in the final semi-solid composition. Preferably, the amount of polyorthoester-compatible liquid is selected from 10, 11, 12, 13, 14, 15, 16, 17, 18, 19, 20, 21, 22, 23, 24, 25, 26, 27, 28, 29, and 30 weight percent. The amount of the granisetron will generally range from about 1-10 weight percent (e.g., 1, 2, 3, 4, 5, 6, 7, 8, 9, and 10 percent by weight). Illustrative amounts further include from about 1-5 weight percent of granisetron. A preferred semi-solid drug delivery vehicle contains 2 weight percent granisetron. A particularly preferred semi-solid drug delivery vehicle is provided in Example 1.

Compositions suitable for use in the instant method include formulations that are bioequivalent to those described herein.

### E. Administration

The semi-solid drug delivery vehicle may be formulated for administration via any suitable route, e.g., oral, transdermal, or by injection (e.g., intradermal, subcutaneous, intramuscular, intravenous, etc.). Generally, the semi-solid drug delivery vehicle is administered by injection, where a preferred route of administration is subcutaneous. Optionally, a diluent may be added to the composition prior to administration, such as saline or sterile water, to assist in delivery. For subcutaneous administration, the semi-solid composition is typically filled into a suitably sized syringe, or a pen, or other suitable injection device, and injected into a patient site that has been determined to be most effective, e.g., arm, leg, or abdomen. Generally, the syringe is fitted with a 16-25 gauge needle, although smaller needles may be used in some embodiments.

Illustrative single dosage amounts of granisetron contained in the semi-solid drug delivery vehicle range from 2 mg to 25 mg. Illustrative single dosage amounts of granisetron contained in the semi-solid drug delivery vehicle include 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, and 25 mg. Preferred dosage amounts as exemplified in Example 2 are 5 and 10 mg of granisetron contained within the semi-solid delivery form. The corresponding amount of the semi-solid delivery vehicle administered can be determined based upon the concentration of granisetron in the semi-solid composition. For example, a single dose of a semi-solid composition containing 2% granisetron for administration of 5 mg of granisetron corresponds to 250 mg of the semi-solid composition, while a single dose of the same composition for administration of 10 mg of granisetron corresponds to 500 mg of the semi-solid composition. Generally, the granisetron is administered at a dosage amount from about 10-250 micrograms/kilogram of patient body mass. Desirably, the amount of granisetron administered is the lowest quantity effective to result in a desirable response, e.g., improvement over the emetic response observed in the case of the previously administered 5-HT3 RA, and more desirably, an absence of emesis, e.g., in the acute stage, in the delayed stage, or in both stages.

Administration of the semi-solid composition, e.g., subcutaneously, typically occurs over a period of, for example, about 60 seconds, or about 30 seconds or about 15 seconds. The period of administration will vary depending upon factors such as the viscosity of the composition, the individual administering the composition, and the like.

The semi-solid composition, according to any one or more of the particular embodiments described herein, is administered to a patient undergoing either high emetogenic risk or moderate emetogenic risk chemotherapy. For the sake of clarity, particular embodiments related to the foregoing are explicitly described below.

For example, a semi-solid granisetron composition as described herein may be administered to a patient undergoing highly emetogenic chemotherapy. Preferably, administration of the semi-solid granisetron composition is effective to provide an improvement (i.e., reduction) in the occurrence of emetic episodes occurring in the acute phase when compared to the previously administered 5-HT3 receptor antagonist when evaluated over the same time frame. Even more preferably, the semi-solid granisetron composition is effective to provide a complete absence of an emetic episode in the acute phase following highly emetogenic chemotherapy. Additionally, in one or more preferred embodiments, administration of the semi-solid granisetron composition is effective to provide an improvement (i.e., reduction) in the occurrence of emetic episodes occurring in the delayed onset phase when compared to the previously administered 5-HT3 receptor antagonist when evaluated over the same time period. Even more preferably, the semi-solid granisetron composition is effective to provide a complete absence of an emetic episode in the delayed onset phase following highly emetogenic chemotherapy.

Alternatively, a semi-solid granisetron composition as described herein may be administered to a patient undergoing moderately emetogenic chemotherapy. Preferably, administration of the semi-solid granisetron composition is effective to provide an improvement (i.e., reduction) in the occurrence of emetic episodes occurring in the acute phase when compared to the previously administered 5-HT3 receptor antagonist when evaluated over the same time period. Even more preferably, the semi-solid granisetron composition is effective to provide a complete absence of an emetic episode in the acute phase following moderately emetogenic chemotherapy. Additionally, in one or more preferred embodiments, administration of the semi-solid granisetron composition is effective to provide an improvement (i.e., reduction) in the occurrence of emetic episodes occurring in the delayed onset phase when compared to the previously administered 5-HT3 receptor antagonist when evaluated over the same time period. Even more preferably, the semi-solid granisetron composition is effective to provide a complete absence of an emetic episode in the delayed onset phase following moderately emetogenic chemotherapy.

The semi-solid composition may be administered prior to chemotherapy, or following administration of the chemotherapeutic agent. Preferably, the semi-solid composition is administered prior to commencement of chemotherapy, e.g., typically within two hours of commencement of chemotherapy. For example, the semi-solid granisetron composition may be administered 1.5 hours prior to commencement of chemotherapy, or 1 hour prior to commencement of chemotherapy, or 45 minutes prior to commencement of chemotherapy or 30 minutes prior to commencement of chemotherapy. Generally, a single dose of the formulation, effective to provide sustained delivery of granisetron, is administered over the course of a single round of chemotherapy to prevent or reduce the occurrence of emetic episodes. For chemotherapies involving a multi-day regimen, such as the 5-day regiment for cis-platin, the semi-solid granisetron composition may be administered only on day 1. Alternatively, the semi-solid granisetron composition may be administered on two of the days over the course of the 5-day treatment regimen. In yet another embodiment, the semi-solid granisetron composition is administered on 3 of the 5 days.

The semi-solid composition is also administered in subsequent cycles of chemotherapy.

In one embodiment of the method, the granisetron-containing semi-solid dosage form provides anti-CINV monotherapy, i.e., is the only anti-emetogenic agent administered.

### F. Supporting Study

In a study conducted in support of the claimed treatment method, the efficacy of a sustained delivery formulation of the 5-HT3 antagonist granisetron, described below, was evaluated in patients receiving chemotherapy who failed to achieve a complete response when previously treated with palonosetron in preventing acute and delayed CINV. As described in Example 2, the patients in the study were undergoing either a moderately emetogenic chemotherapy regimen or a highly emetogenic chemotherapy regimen. In a first cycle of the study, the patients were treated with (i) 5 mg of granisetron in a semi-solid drug delivery vehicle, administered subcutaneously; (ii) 10 mg of granisetron in a semi-solid drug delivery vehicle, administered subcutaneously; or (iii) palonosetron, 0.25 mg administered intravenously. Patients who received palonosetron in Cycle 1 and remained on study were re-randomized for treatment with 5 mg or 10 mg of granisetron, administered as comprised within a semi-solid drug delivery vehicle via subcutaneous injection of 250 mg vehicle or 500 mg vehicle. The complete response rates in Cycle 2 were assessed for patients receiving the 500 mg of the granisetron-containing semi-solid drug delivery vehicle who did not achieve a complete response in Cycle 1 with palonosetron.

The results are shown in Table 1 below and discussed in detail in Example 2. The granisetron-containing semi-solid drug delivery vehicle demonstrated substantial efficacy (i.e, a complete response) in patients receiving MEC or HEC who had previously failed treatment with palonosetron. Accordingly, failure to achieve an initial complete response to palonosetron at a recommended 0.25 mg intravenous dose is in no way predictive of failure of a granisetron-containing semi-solid drug delivery vehicle in subsequent HEC or MEC cycles.

TABLE 1

| | Cycle 2 | | Acute | | Delayed | | | |
|---|---|---|---|---|---|---|---|---|
| | (failed | Cycle 1 → | CINV | | CINV | | Overall | |
| Cycle 1 | palonosetron) | Cycle 2 | N | % | N | % | n | % |
| MEC (n = 208) | granisetron, | All Failures | 19 | | 34 | | 38 | |
| palonosetron | SQ delivery | Fail → Fail | 8 | | 21 | | 23 | |
| | vehicle | Fail → CR | 11 | 57.9 | 13 | 38.2 | 15 | 39.5 |
| | (n = 38) | | | | | | | |
| HEC (n = 238) | granisetron, | All Failures | 12 | | 33 | | 34 | |
| palonosetron | SQ delivery | Fail → Fail | 5 | | 18 | | 20 | |
| | vehicle | Fail → CR | 7 | 58.3 | 15 | 45.5 | 14 | 41.2 |
| | (n = 34) | | | | | | | |

It will be appreciated that the treatment method described herein contemplates administration of a granisetron-containing drug delivery vehicle to an individual experiencing CINV and previously treated with any 5-HT3 antagonist other than granisetron. The study described herein demonstrated that patients previously treated with palonosetron and who did not respond to palonosetron to prevent or treat CINV, were responsive to a granisetron-containing drug delivery vehicle. A skilled artisan will appreciate that the findings herein are equally applicable to patients previously treated with, but failed to respond to, a selective 5-HT3 receptor antagonist other than granisetron, such as but not limited to ondansetron, dolasetron, tropisetron, and palonosetron.

### EXAMPLES

The following examples are illustrative in nature and are in no way intended to be limiting.

### Example 1

### Preparation of Semi-Solid Delivery Vehicle

A pharmaceutical composition comprising 2% granisetron and a semi-solid delivery vehicle comprised of the polyorthoester detailed below was prepared:

(i) 78.4 weight % of the polyorthoester of formula I:

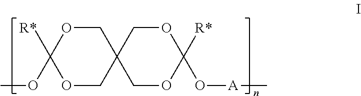

where:

R* is a C2 alkyl;

n is an integer of at least 5; and

A is R1 or R3 where R1 is:

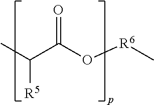

where:

p is on average 2, or varies between 1-20; R5 is hydrogen; and

R6 is:

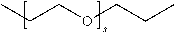

where:

s is 3; and R3 is:

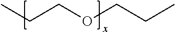

where x is 3;
where the polyorthoester comprises 42.9 mole % DETOSU, 38.1 mole % TEG, and 19.1 mole % of the A units are of the formula R1; and
(ii) a pharmaceutically acceptable, polyorthoester-compatible liquid excipient that is 19.6 weight % MPEG 550 (methoxy-polyethylene glycol, Mn 550).

More specifically, the semi-solid drug delivery vehicle containing 2 weight percent granisetron was prepared as described in U.S. Pat. No. 8,252,305, Example 2 (c). The composition contained 78.4 weight percent polyorthoester (prepared by reaction of DETOSU (3,9-di(ethylidene)-2,4,8,10-tetraoxaspiro[5.5]undecane), triethylene glycol (TEG), and triethylene glycol diglycolide (TEG-diGL) at the following molar ratio: 90:80:20), 19.6 weight percent polyethylene glycol monomethyl ether 550 (mPEG550), and 2 weight percent granisetron. Thus, administration of a 250 mg dose of the semi-solid delivery vehicle is equivalent to administration of 5 mg granisetron, while administration of a 500 mg dose of the semi-solid delivery vehicle is equivalent to administration of 10 mg of granisetron.

### Example 2

### Treatment with Granisetron in Semi-Solid Delivery Vehicle

In Cycle 1 of the study, 1395 patients receiving single doses of a moderately emetogenic chemotherapy (MEC) regimen or a highly emetogenic chemotherapy (HEC) regimen were randomized for treatment with one of three regimens: a semi-solid drug delivery vehicle comprising granisetron, administered subcutaneously to provide (i) 5 mg granisetron or (ii) 10 mg granisetron (via subcutaneous injection of 250 mg or 500 mg vehicle, respectively) or (iii) palonosetron, 0.25 mg intravenous. The palonosetron dose administered was the recommended dose of palonosetron (ALOXI®) for treatment of chemotherapy-induced nausea and vomiting in adults. Palonosetron, when administered intravenously at the above-dose, is indicated in adults for the prevention of both acute and delayed vomiting associated with initial and repeated courses of moderately emetogenic chemotherapy, and acute nausea and vomiting associated with initial and repeat courses of highly emetogenic chemotherapy.

Patients who received palonosetron in Cycle 1 and remained in the study were re-randomized for treatment in Cycle 2 with 5 mg or 10 mg granisetron administered subcutaneously as 250 mg or 500 mg, respectively, of the semi-solid drug delivery vehicle. Complete response rates in Cycle 2 were assessed for patients receiving 500 mg of the subcutaneous granisetron delivery vehicle who did not achieve complete response in Cycle 1 with palonosetron. Complete response was defined as the absence of emetic episodes over the appropriate time period (0 to 24 hours after chemotherapy for acute; 24 to 120 hours after chemotherapy for delayed onset).

Results: 446 patients received palonosetron in Cycle 1 (208 MEC; 238 HEC). Of these, 194 (43.5%) were overall (0-120 hour) failures (100/208 [48.1%] MEC; 94/238 [39.5%] HEC). Of 194 Cycle 1 palonosetron failures, 72 were re-randomized prior to Cycle 2 to 500 mg of the granisetron-containing semi-solid delivery vehicle (38 MEC; 34 HEC). Of 38 MEC palonosetron failures who received 500 mg of the granisetron-containing semi-solid delivery vehicle in Cycle 2, overall complete response was 39.5% (57.9% acute; 38.2% delayed). Of 34 HEC palonosetron failures who received 500 mg of the granisetron-containing semi-solid delivery vehicle in Cycle 2, overall complete response was 41.2% (58.3% acute; 45.5% delayed). In the acute phase, greater than 50% of MEC and HEC patients who failed palonosetron in Cycle 1 achieved complete response to 500 mg of the granisetron-containing semi-solid delivery vehicle in Cycle 2. Complete response rate for patients receiving HEC was slightly less in the delayed versus acute setting.

These data demonstrate the surprising efficacy of the 5-HT₃ antagonist, granisetron, when administered via a polyorther-ester based semi-solid delivery vehicle, to effectively treat CINV in patients that were unresponsive to treatment with another 5-HT3 antagonist, palonosetron, when administered at a recommended dosage.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A treatment method, comprising:
administering to a patient receiving chemotherapy, a semi-solid drug delivery vehicle comprising a bioerodible polymer and granisetron, wherein the patient was previously treated with a 5-HT₃ antagonist other than granisetron and failed to achieve a satisfactory prevention or reduction of acute or delayed chemotherapy-induced nausea and vomiting.

2. The method of claim 1, wherein the semi-solid drug delivery vehicle is administered subcutaneously.

3. The method of claim 1, where the patient that failed to achieve a satisfactory prevention or reduction of acute or delayed chemotherapy-induced nausea and vomiting was previously treated with palonosetron.

4. The method of claim 3, where the patient that failed to achieve a satisfactory prevention or reduction of acute or delayed chemotherapy-induced nausea and vomiting was previously treated with intravenously administered palonosetron.

5. The method of claim 3, wherein the patient is undergoing treatment for acute chemotherapy-induced nausea and vomiting.

6. The method of claim 3, wherein the patient is undergoing treatment for delayed onset chemotherapy-induced nausea and vomiting.

7. The method of claim 5, wherein the patient is receiving moderately emetogenic chemotherapy.

8. The method of claim 5, wherein the patient is receiving highly emetogenic chemotherapy.

9. The method of claim 7, comprising administering to the patient a single dose of the semi-solid drug delivery vehicle comprising from 1 to 25 mg of granisetron during one cycle of chemotherapy.

10. The method of claim 9, wherein the single dose of the semi-solid drug delivery vehicle comprises 5 or 10 mg of granisetron.

11. The method of claim 9, wherein the single dose is administered prior to commencement of chemotherapy.

12. The method of claim 9, wherein the single dose is administered post-chemotherapy.

13. The method of claim 3, wherein granisetron is the only anti-emetic agent comprised within the semi-solid drug delivery vehicle.

14. The method of claim 3, effective to provide a measurable prevention or reduction of acute or delayed chemotherapy-induced nausea and vomiting when compared to previous treatment with palonosetron.

15. The method of claim 14, effective to result in a complete absence of an emetic episode in the acute phase.

16. The method of claim 14, effective to result in a complete absence of an emetic episode in the delayed phase.

17. The method of claim 14, effective to result in a complete absence of an emetic episode in both the acute and delayed phase following chemotherapy.

18. The method of claim 3, wherein the administering is continued over one or more additional rounds of chemotherapy.

19. The method of claim 1, wherein the bioerodible polymer is a polyorthoester.

20. The method of claim 19, wherein the polyorthoester comprises subunits selected from 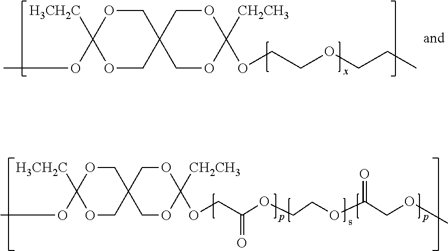
where
x is an integer selected from 1, 2, 3, and 4,
the total amount of p is an integer selected from 1, 2, 3, 4, 5, 6, 7, 8, 9, 10, 11, 12, 13, 14, 15, 16, 17, 18, 19 and 20,
s is an integer selected from 1, 2, 3, and 4,
the mole percentage of α-hydroxyacid containing subunits in the polyorthoester is from about 0.1 to about 25 mole percent,
and the polyorthoester has a molecular weight in a range of about 1000 to 10,000.

21. The method of claim 1, wherein the semi-solid drug delivery vehicle further comprises an excipient selected from polyethylene glycol ether derivatives having a molecular weight between about 200-4,000.

22. The method of claim 21, wherein the polyethylene glycol ether derivative is polyethylene glycol monomethyl ether 550.

23. The method of claim 19, wherein the semi-solid drug delivery vehicle comprises a polyorthoester, about 10-50 weight percent polyethylene glycol monomethyl ether having a molecular weight in a range of about 200 to 4,000, and about 1-5 weight percent granisetron.

24. The method of claim 23, wherein the polyorthoester is a reaction product of 3,9-di(ethylidene)-2,4,8,10-tetraoxaspiro[5.5]undecane (DETOSU), triethylene glycol and triethylene glycol diglycolide.

25. The method of claim 24, wherein the mole percentage of glycolide-containing subunits in the polyorthoester is from about 0.1 to about 25 mole percent.

26. The method of claim 23, wherein the semi-solid drug delivery vehicle comprises from about 70-80 weight percent polyorthoester, about 15-25 weight percent polyethylene glycol monomethyl ether and from 1 to 5 weight percent granisetron.

27. (canceled)

28. (canceled)
